(19) United States
(12) Patent Application Publication
(10) Pub. No.: US 2024/0344103 A1
(43) Pub. Date: Oct. 17, 2024

(54) IN VITRO MODELS FOR ESTIMATING DRUG DOSAGE

(71) Applicant: Hennepin Healthcare Research Institute, Minneapolis, MN (US)

(72) Inventors: Ajay K. Israni, Chanhassen, MN (US); Guillaume C. Onyeaghala, Minneapolis, MN (US)

(21) Appl. No.: 18/682,534

(22) PCT Filed: Aug. 10, 2022
(86) PCT No.: PCT/US2022/039966

§ 371 Date: Feb. 9, 2024

Related U.S. Application Data
(60) Provisional application No. 63/232,369, filed on Aug. 12, 2021.

(51) Int. Cl. C12Q 1/02; C12N 1/20

(52) U.S. Cl. CPC C12Q 1/025; C12N 1/20; C12Q 2600/106; C12Q 2600/156

(57) ABSTRACT

A method for evaluating metabolism of an active compound includes providing a composition comprising a microbiome obtained from a subject that has received an active compound for a period of time sufficient to achieve steady state; forming a mixture of the composition with either the active compound or a deactivated metabolite of the active compound; identifying a change in the mixture, comprising a reduction in the amount of the active compound or deactivated metabolite, presence of the active compound modified by the microbiome or a re-activated active compound; estimating, from the change in the mixture, the ability of the subject's microbiome to process the active compound or deactivated metabolite in the subject and affect the concentration of the active compound in the subject; determining a dosage required to achieve a concentration of the active compound in the subject that is therapeutically effective; and administering to the subject the dosage required.

### CROSS-REFERENCE TO RELATED APPLICATION

This application claims the benefit of U.S. Provisional Patent Application No. 63/232,369, filed Aug. 12, 2021, which is incorporated herein by reference in its entirety.

### GOVERNMENT FUNDING

This invention was made with government support under AI140303 awarded by the National Institutes of Health. The government has certain rights in the invention.

### SUMMARY

This disclosure describes, in one aspect, a method for evaluating metabolism of an active compound. Generally, the method includes providing a composition comprising a microbiome, wherein the microbiome is obtained from a subject that has received an active compound for a period of time sufficient to achieve steady state; combining a deactivated metabolite of the active compound and the composition to form a mixture; incubating the mixture; identifying a change in the mixture, wherein the change comprises a reduction in the amount of the deactivated metabolite, presence of a re-activated active compound, or both; estimating, from the change in the mixture, the ability of the subject's microbiome to process the deactivated metabolite in the subject and affect the concentration of the active compound in the subject; determining a dosage required to achieve a concentration of the active compound in the subject that is therapeutically effective considering effects of the subject's microbiome on the concentration of the active compound in the subject; and administering to the subject the dosage required to achieve a concentration of the active compound in the subject that is therapeutically effective.

In one or more embodiments, the microbiome is a human microbiome.

In one or more embodiments, the active compound is a pro-drug.

In one or more embodiments, the metabolite is a metabolite of a drug or a metabolite of a pro-drug.

In one or more embodiments, the method further includes obtaining an abbreviated AUC, a trough concentration, or a combination thereof, from the subject, wherein the estimating comprises using the change in the mixture and either the abbreviated AUC, the trough concentration, or the combination thereof.

In one or more embodiments, the active compound is a compound that is inactivated by the liver.

In one or more embodiments, the method further includes screening a sample from the subject to detect the presence or absence of a polymorphic variant of a coding region encoding at least one of a hepatic enzyme, a transporter, or a combination thereof; and determining variation in metabolism of the active compound by the at least one hepatic enzyme, transporter, or a combination thereof.

In another aspect, this disclosure describes a composition that generally includes a microbiome obtained from a subject that has received an active compound for a period of time sufficient to achieve steady state and a deactivated metabolite of the active compound.

In another aspect, this disclosure describes a method for evaluating metabolism of a compound. Generally, the method includes providing a composition comprising a microbiome, wherein the microbiome is obtained from a subject that has received an active compound for a period of time sufficient to achieve steady state; combining the active compound and the composition to form a mixture; incubating the mixture; identifying a change in the mixture, wherein the change comprises a reduction in the amount of the active compound, presence of the active compound modified by the microbiome, or both; estimating from the change in the mixture the ability of the subject's microbiome to process the active compound in the subject and affect the concentration of the active compound in the subject; determining a dosage required to achieve a concentration of the active compound in the subject that is therapeutically effective considering effects of the subject's microbiome on the concentration of the active compound in the subject; and administering to the subject the dosage required to achieve a concentration of the active compound in the subject that is therapeutically effective.

In one or more embodiments, the microbiome is a human microbiome.

In one or more embodiments, the active compound is a drug that is absorbed in the gastro-intestinal tract.

In one or more embodiments, the active compound is a drug administered orally and is modified by the microbiome prior to absorption by intestinal cells.

In one or more embodiments, the method further includes obtaining an abbreviated AUC, a trough concentration, or a combination thereof, from the subject, wherein the estimating comprises using the change in the mixture and either the abbreviated AUC, the trough concentration, or the combination thereof.

In another aspect, this disclosure describes a composition that generally includes a microbiome obtained from a subject that has received an active compound for a period of time sufficient to achieve steady state; and the active compound.

In another aspect this disclosure describes a method for identifying a subject as a weak responder to an active compound. Generally, the method includes administering the active compound to the subject for a period of time sufficient to achieve steady state; detecting cells of the subject's microbiome that contain the active compound; and identifying the subject as a weak responder to the active compound.

In another aspect, this disclosure describes a method for identifying a subject as a strong responder to an active compound. Generally, the method includes administering the active compound to the subject for a period of time sufficient to achieve steady state; detecting cells of the subject's microbiome that contain lower than typical amount of the active compound; and identifying the subject as a strong responder to the active compound.

In another aspect, this disclosure describes a method that generally includes providing cells containing an active compound and lysing the cells, thereby releasing the active compound.

In one or more embodiments, the cells include microbiome cells from a subject that has received the active compound for a period of time sufficient to achieve steady state.

In another aspect, this disclosure describes a method that generally includes providing cells containing an active compound and contacting the cells with a compound that induces the cells to release the active compound.

In one or more embodiments, the cells include microbiome cells from a subject that has received the active compound for a period of time sufficient to achieve steady state.

In one or more embodiments, the compound that induces the cells to release the active compound comprises a metabolic substrate of the cells.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1. In vivo metabolism of mycophenolate mofetil. The major metabolic pathway of mycophenolic acid (MPA), the active form of mycophenolate mofetil. Beta-glucuronidase is important in the conversion of MPAG back to MPA in the gut and EHR.

FIG. 2. A general process of the in vitro culture associated with MPA drug metabolism. The assay includes one step executed in an anaerobic environment to approximate the drug metabolism in vivo by the microbiome, and subsequent quantitation and optional microbiome sequencing steps to provide personalized dosing of a drug to a patient based on their microbiome metabolism.

FIG. 3. A sample from a kidney transplant patient having received treatment with mycophenolate mofetil for over a month. A random stool sample was collected around a 12-hour pharmacokinetic visit. Mycophenolate mofetil (MMF) is actively converted to its active component MPA in the body. The figure describes an experiment in which gut microbiota from the random stool sample were exposed to MPA in an in vitro setting. MPA concentrations are presented as mean concentrations from the triplicate samples and their 95% confidence intervals. Compared to the control samples, MPA concentrations did not change in after incubation with the stool bacteria, and remained fairly stable at six hours, 24 hours, and 48 hours post incubation. The control samples have no stool sample in them.

FIG. 4. A sample from a kidney transplant patient having received treatment with mycophenolate mofetil for over a month. A random stool sample was collected around a 12-hour pharmacokinetic visit. A proof of concept is provided with MPAG conversion to MPA by gut microbiota to approximate the area under the curve seen in vivo due to EHR. The data show the rapid metabolism of MPAG by stool microbiota under anaerobic conditions in the first six hours post incubation. This decrease in MPAG is concurrent with an increase in MPA concentrations in the same samples, with an ongoing upward trend in the MPA concentration observed at 24 and 48 hours post incubation. The MPA concentration is higher than the amount expected if all MPAG is converted to MPA. This supports the concept that MPA from within the microbial cells was released by adding MPAG substrate to the in vitro assay.

FIG. 5. Rapid metabolism of MPAG to MPA by gut microbiota under anaerobic conditions in yeast extract-casein hydrolysate-fatty acids (YCFA) media in the first six hours post incubation. Stool samples from five kidney transplant patients being treated with mycophenolate mofetil for longer than one month were used. A random stool sample was collected around a 12-hour pharmacokinetics visit (FIG. 6). The conversion of mycophenolic acid glucuronide (MPAG) to mycophenolic acid (MPA) by gut microbiome at six hours correlates with percentage enterohepatic recirculation (EHR) in vivo [correlation coefficient=0.82 (95% CI—0.21 to 0.98, P=0.08). Percent EHR defined as MPA AUC6-12 hour/AUC₀-12 hour×100.]. These participants were considered to have high MPAG to MPA conversion.

FIG. 6. Mycophenolic acid (MPA) 12-hour concentration time-profile after administration, from five study participants. Calculated mean MPA trough (SD) was 3.5 mg/L (±1.66), mean dose normalized MPA AUC0-12 was 0.07 h-mg/L (±0.02), and % EHR was 46% (+8 range 41-60%). Percent EHR was defined as MPA AUC6-12 hour/AUC₀-12 hour×100.

FIG. 7. Repeat assay of mycophenolic acid glucuronide (MPAG) conversion to mycophenolic acid (MPA) by gut microbiome at six hours and 24 hours. Stool samples from three kidney transplant patients with being treated with mycophenolate mofetil for longer than one month were used. These participants were considered to have high MPAG to MPA conversion and their stool samples, which had been frozen at −80° C. for a mean of 312 days (range 246-385 days), thawed at 4° C. overnight, then used for a repeat in vitro assay at 37° C.

FIG. 8. Quantification of MPA and MPAG in the supernatant of a mixture of YCFA and stool microbiota of eight patients (N=8 independent samples, n=5 technical replicates for each participant). These samples were lysed using four different combinations of physical disruption (3 cycles of 90 seconds of bead beating) with additives to facilitate cell lysis (750 μL of POWERBEAD solution (Qiagen, Hilden, Germany)+60 μL of C1 solution per the POWERSOIL (Qiagen, Hilden, Germany) microbiome protocol, 200 pg/ml of lysozyme+100 μL of proteinase inhibitor, or 10 mL of hypotonic solution+100 μL of proteinase inhibitor).

FIG. 9. Relationship between a hypothetical 12-hour AUC curve, abbreviated AUC used in clinical care, and the MPA regeneration seen in vitro. Abbreviated 2-hour and 4-hour AUCs and trough concentrations only capture the first MPA peak seen over the lifetime of the drug in the body. The in vitro model demonstrates how the stool microbiota regenerate the MPA molecule from the deactivated MPAG metabolite, which results in the second MPA peak seen in the 12-hour AUC assay.

FIG. 10. Regional plot of chromosome 1 indicating SNP rs7544655, which is most strongly associated with MPA trough levels at day 70 post kidney transplant.

### DETAILED DESCRIPTION OF ILLUSTRATIVE EMBODIMENTS

This disclosure describes methods for analyzing the pharmacokinetics of compounds in the body of a subject. In some embodiments, the methods can be used as a supplement to, or a replacement of, estimating the pharmacokinetics of compounds using standard methods such as an abbreviated AUC or trough concentration.

The methods provided by the present disclosure measure the ability of a patient's microbiome to metabolize a compound. As used herein, the term “microbiome” and “microbiota” are used interchangeably and refer to a collection of microbes present in, or obtained from, a subject's stool. By measuring the ability of a subject's microbiome to metabolize a compound, one can estimate metabolism of the compound in vivo by the microbiome, and that estimate can be used for determining the dose of a drug that is appropriate for the patient. The information gained from the in vitro microbiome sample may be further useful for providing guidance regarding the degree to which the dose should be adjusted to achieve and/or maintain an effective dose that is not so high as to be toxic and not so low as to be sub-therapeutic. In one or more embodiments, further identification of the genetics of hepatic enzymes and transporters of the patient in combination with metabolism by an in vitro microbiome culture can approximate the overall drug metabolism of the drug, and thus allow for more precise adjustment of dosage to the patient.

Active compounds such as drugs (including inactive form such as pro-drugs), pharmaceutical agents, dietary compounds, dietary supplements, industrial chemicals, and industrial pollutants are metabolized by microbes within the body of an individual and eventually eliminated. For instance, some drugs that are administered orally are modified by the microbiome and then absorbed by intestinal cells. Drug modifications performed by the microbiome include, but are not limited to, activation, de-activation, or toxification. Many other drugs, whether delivered orally or by some other route, are inactivated by the liver by enzymes such as glucuronosyltransferases (UGT) enzymes or cytochrome P450, pumped into bile ducts by proteins such as ABCB1 and transported to the small intestine. In the small intestine some inactivated drugs can be reactivated by the microbiome and absorbed back into the circulation to start the process again. This process of transport from liver to intestine, reactivation in the intestine, and transport back into the liver is referred to as enterohepatic recirculation (EHR) and can contribute to prolonging the exposure of a subject to a drug. Since under-dosing a drug can lead to reduced efficacy and over-dosing can lead to toxicity, accurate drug dosing often involves considering the contribution of EHR to the concentration of a drug. Existing methods for determining the contribution of EHR and altering drug dosage exist but are logistically difficult to use in an outpatient, clinical setting. This disclosure provides methods that can supplement to replace estimating the pharmacokinetics of compounds using existing standard methods.

### Compositions

The methods of the present disclosure include providing a composition that includes a microbiome. In one or more embodiments, the microbiome is obtained from a patient that is receiving an active compound to treat a condition. Examples of conditions include but are not limited to cancer (e.g., where the patient is receiving an anti-cancer compound) and recuperation from a transplant (e.g., where the patient is receiving an immunosuppressant to prevent rejection of the transplanted tissue). Exemplary compounds include, but are not limited to, a drug, a pharmaceutical agent, a dietary compound, a dietary supplement, an industrial chemical, or an industrial pollutant that is metabolized by microbes within the body of an individual and eventually eliminated. In one or more embodiments, the active compound is metabolized by intestinal microbes and is not providing a desired degree of therapeutic effect despite usual dosing.

Typically, the patient is one to whom the active compound has been administered for a period of time sufficient to allow a steady state of the active compound in the body to be achieved. This helps to ensure that the patient's microbiota has reached steady state and is actively metabolizing the active compound. A human microbiome stool sample may be collected at any time during the steady state period for use in the method. Whether steady state has been achieved in a patient can be determined by considering the half-life of the active compound. Typically, steady state is reached in a patient after a period of time equivalent to at least two half-lives, at least 2.5 half-lives, at least three half-lives, or at least 3.5 half-lives of the active compound in a body has elapsed. The half-life of an active compound used in therapy is often available from the manufacturer or can be determined using routine methods.

The composition further includes components suitable for culturing the microbiome sample. Microbiological media useful for culturing microbiome samples are known to the person skilled in the art and include, but are not limited to, YCFA (Yeast Extract-Casein Hydrolysate-Fatty Acids) medium. Typically, the conditions are suitable for culture of anaerobic microbes. The skilled person will recognize that a culture of a microbiome sample as described herein does not necessarily represent all microbes present in the original sample, because some microbes present in a human's large gastrointestinal tract are not culturable.

### Active Compounds

The methods further include combining the microbiome composition with an active compound to produce a mixture. The active compound can be any active compound to which a human can be exposed, enters EHR, and is eventually reabsorbed by intestinal cells and introduced back into the human's circulatory system. Examples of active compounds include, but are not limited to, drugs, pharmaceutical agents, dietary compounds, industrial chemicals, industrial pollutants, dietary supplements, and metabolites of any compound in the preceding types of active compounds. In one or more embodiments, the active compound is an active or an inactive form of a drug (e.g., a biologically active compound that has a therapeutic benefit under certain conditions). Thus, the term “active compound” expressly includes inactive form of a drug such as a pro-drug (e.g., a biologically inactive compound that can be metabolized in the body to produce a drug) or an inactive form of a drug (e.g., a metabolite of a drug, including a metabolite that has been inactivated by hepatic enzymes). In one or more embodiments, the active compound used in the method is one that enters the EHR system after processing by the liver and is then processed by the microbiome to yield a compound relevant in identifying and adjusting a therapeutically effective dose of an active compound in the patient. Non-limiting examples of drugs that enter the EHR system and are metabolized by the gut microbiome are listed in Table 1. This non-limiting list of drugs is organized by the effect of the gut microbiome on the active compound (leading to either toxicity, activation, or inactivation of the compound introduced into the intestine by EHR).

TABLE 1

| | | | Effect of |
|---|---|---|---|
| | | Gut Microbiota | microbial |
| Drug Name | Function | Modification | modification |
| Irinotecan | Cancer therapy drug | De-glucuronidation of the | Toxicity [1, 2] |
| | | active metabolite in the gut | |
| Indomethacin | Anti-inflammatory drug | Beta-glucuronidase driven | Toxicity [2, 3] |
| | | cleavage of NSAID glucuronide | |
| Diclofenac | Anti-inflammatory drug | Beta-glucuronidase driven | Toxicity [2, 3] |
| | | cleavage of NSAID glucuronide | |
| Ketoprofen | Anti-inflammatory drug | Beta-glucuronidase driven | Toxicity [2, 3] |
| | | cleavage of NSAID glucuronide | |
| Mycophenolate | Immunosuppressive drug | Gut microbiota metabolize MPA | Toxicity [4-6] |
| Mofetil (MMF) | | glucuronide (MPAG) back to | |
| | | mycophenolic acid (MPA) | |
| Sulfasalazine | Inflammatory bowel disease | Gut microbiota reduce sulfasalazine | Toxicity [2, 4-6] |
| | (IBD) medication | into sulfapyridine and the active | |
| | | anti-inflammatory agent 5-ASA | |
| Sulindac | Anti-inflammatory drug | De-glucoronidation of the | Toxicity [2, 4-6] |
| | | active metabolite in the gut | |
| Loperamide | Anti-diarrheal drug | De-glucoronidation of the | Toxicity [2, 4-6] |
| | | active metabolite in the gut | |
| Levodopa | Parkinson's disease | Microbial decarboxylation and | Inactivation [2] |
| | therapeutic drug | p-dehydroxylation convert L-dopa | |
| | | to m-tyramine | |
| Cladribine | Cancer therapy drug | Inhibited efficacy | Inactivation[7] |
| Gemcitabine | Cancer therapy drug | Inhibited efficacy | Inactivation[7] |
| Digoxin | Congestive heart failure | Eggerthella lenta metabolizes | Inactivation[2] |
| | therapy drug | digoxin to dihydrodigoxin | |
| Tacrolimus | Immunosuppressive drug | Specific organisms metabolize | Inactivation [8] |
| | | Tacrolimus to a less effective | |
| | | metabolite | |
| Benzylpenicillin | Cancer therapy drug | Beta-lactamase inhibition | Inactivation[9] |
| Ipilimumab | Antil CTLA-4 human | B. thetaiotaomicron and B. Fragilis | Activation [2, 10] |
| | monoclonal antibody (Ab) | enhance T-cell response to antibodies | |
| | therapeutic drug | | |
| Metformin | Type 2 diabetes (T2D) | Akkermansia muciniphila abundance | Activation [2, 11] |
| | therapy drug | is modulated by metformin treatment | |
| Fludarabine de | Cancer therapy drug | Improved efficacy | Activation [7] |
| phosphate | | | |
| CB1954 | Cancer therapy drug | Improved efficacy | Activation [7] |

1. Wallace et al., Science 2010, 330: 831-835.
2. Spanogiannopoulos et al., Nat Rev Microbiol 2016, 14: 273-287.
3. Saitta et al., Xenobiotica 2014, 44: 28-35.
4. Biernat et al., Sci Rep 2019, 9: 825.
5. Pellock et al., J Mol Biol 2019, 431: 970-980.
6. Wallace et al., Chem Biol 2015, 22: 1238-1249.
7. Lehouritis et al., Sci Rep 2015, 5: 14554.
8. Guo et al., Drug Metab Dispos 2019, 47: 194-202.
9. Hennessey, J Gen Microbiol 1967, 49: 277-285.
10. Vetizou et al., Science 2015, 350: 1079-1084.

In one or more embodiments, the active compound added to a microbiome composition includes a drug metabolite or inactive drug to determine the re-activation of the inactive drug or metabolite. In one or more embodiments, the active compound added to a microbiome composition includes the parent drug or active drug to determine its de-activation.

The amount of active compound added to the microbiome composition is set as a standard dose in order to measure the degree of activation or deactivation by the microbiome from the microbiome composition. The amount of active compound added is within the detectable range of the certified reference material used for quantifying the active compound when added to the microbiome composition. This detectable range of the certified reference material is used as internal reference standard for quantifying the active compound. The range of certified reference material used for quantification of an active compound varies depending on the method used for detection, and the skilled person will recognize this information is provided by the entity that created the certified reference material. Typically, the amount of the active compound is in an amount sufficient to identify an increase or decrease in the concentration after processing by the microbiome, and identification of the rate of change of the active compound by the microbiome. In one or more embodiments, the amount of active compound is at least 0.1-fold, 0.5-fold, 1-fold, 1.5-fold, 2-fold, 2.5-fold, 3-fold, 3.5-fold, 4-fold, 4.5-fold, or greater than the concentration provided as a certified reference material to serve as internal standard when quantifying the active compound.

### Identifying a Processed Compound

After combining the active compound with the microbiome composition, the cultures are incubated for various times, and then assayed to determine the presence and amount of a compounds of interest—e.g., the active compound added to the microbiome composition and/or the metabolite compound that results after the active compound is processed by the microbiome. The incubation times can be, for instance, less than one hour (e.g., minutes or seconds), one hour, two hours, four hours, six hours, 12 hours, 24 hours, 36 hours, 48 hours, or longer. The sample is processed at the appropriate time point by separating the microbes and other solids from the rest of the composition, typically by centrifugation, and the supernatant is used in an analytical method to identify the active compound and/or metabolite compound.

Any suitable analytical method can be used to identify the compound of interest, and the method can depend on the compound of interest to be identified. Exemplary analytical methods include, but are not limited to, high performance liquid chromatography (HPLC) and enzyme multiplied immunoassay technique. The skilled person will recognize that use of these and other analytical techniques can be easily applied to the analysis of any compound, including but not limited to identifying the version of the compound that is transported by EHR to the intestine and identifying the version of the compound that results after metabolism by the microbiome.

Correlating metabolism by microbiome to the amount of active compound present in a patient The amount of active compound present in the patient can be determined using an area under the curve (AUC) analysis with several blood samples drawn during the time the drug undergoes EHR. However, this AUC measurement requires a specialized research stay and is not logistically feasible in an outpatient, routine clinical setting. Accordingly, trough-concentration measurements and/or an abbreviated AUC is conducted in routine clinical setting. While these measurements are very helpful in establishing dosage, they are among the only methods available and can lead to conclusion based on insufficient information. Using the composition described herein that includes components suitable for culturing the microbiome sample from the patient undergoing an AUC measurement along with the form of the active compound processed into the bile and then moved into the small intestine, greater understanding of processing of the compound by the specific patient is possible. A higher amount of active compound generated by this microbiome composition predicts a higher amount of active compound circulated by EHR and a higher amount of active compound present in the patient undergoing an AUC measurement. Likewise, a lower amount of active compound generated by this microbiome composition predicts a lower amount of active compound circulated by EHR and lower amount of active compound in a patient undergoing an AUC measurement. This information can be used by a physician to alter dosage and aid patient treatment and survival.

For instance, a kidney transplant patient is usually administered multiple compounds to aid in immunosuppression to prevent transplant rejection. If a kidney transplant patient is experiencing toxicity such as low blood count or low white cell count, it is very difficult to predict which active compound (or active compounds) is causing the toxicity. The in vitro method described herein can be conducted for one or more of the active compounds. The generation of a higher amount of active compound by this culture predicts a higher amount of active compound circulated by EHR and a higher amount of active compound present in the patient. In this case, the physician can make an informed decision to decrease the dose of this specific compound alleviate the toxicity.

Conversely, a patient may experience an acute rejection, indicating that one or more of the immunosuppressive drugs should be increased. It can be difficult to predict which immunosuppressive active compound (or immunosuppressive active compounds) should be increased to ameliorate the risk of acute rejection. Moreover, increasing dosage of an active compound that is near the concentration that induces toxicity can lead to other undesirable outcomes. The in vitro method described herein can be used here to identify one or more immunosuppressive active compounds whose dosage should be increased to address the acute rejection while reducing the likelihood of inducing toxicity by increasing the dose of an active compound already at or near the concentration that induces toxicity. Generating lower amounts of an active compound by the culture predicts a lower amount of active compound circulated by EHR and lower amount of active compound in the patient. In this case, the physician can make the informed decision to increase the dose of this specific active compound and reduce risk of recurrent rejection.

The skilled person will recognize that the results from using the in vitro method of the present disclosure can be combined with any trough-concentration and/or abbreviated AUC data to better establish an understanding of the pharmacokinetics of a compound in a patient.

### Genotyping

The method can further include identifying the genotype of the patient. Certain hepatic enzymes can influence the degree to which some compounds are processed by a patient's liver, and thereby cause EHR to have a greater than normal or less than normal effect on the amount of a compound re-entering a patient's body. Next Generation Sequencing methods can be used to easily and quickly perform targeted sequencing of specific genes that encode hepatic enzymes and/or transporters and identify genetic variants with high or low activity. Examples of genes encoding hepatic enzymes and transporters include, but are not limited to, cytochrome P450 proteins, UGT1A8/9, ABCB1, UGT2B7, SLCO1B1, and MRP-2. Genetic variants in hepatic enzymes and transporters determine, at least in part, variability in the inactivation of the compound by the liver and/or transport to the small intestines. Some hepatic enzyme variants affect variability in the absorption of the activated compound into the circulation after it has been metabolized by the microbiome. Thus, the combination of information regarding genetic variants with the information predicting EHR allows one to predict variability in the amount of active compound in a patient undergoing an AUC measurement.

Accordingly, the method can optionally include screening a sample from a subject and detecting the presence or absence of a polymorphic variant of one or more hepatic enzymes and/or one or more transporters. The detecting can include determining if an identified polymorphic variant encodes a protein having a high or low activity regarding the processing of a therapeutic compound, deactivated metabolite, or drug.

For instance, if there is evidence a patient has one or more enzymes and/or transporters that are associated with increasing metabolism of a compound into an inactive metabolite, the physician can make an informed decision to increase the dose of the compound. Likewise, if the results of the in vitro assay with the patient's microbiome predict a decrease in EHR, the physician can make an informed decision to further increase the dose of the compound.

Conversely, if there is evidence a patient has one or more enzymes and/or transporters that are associated with decreasing metabolism of a therapeutic compound into an inactive metabolite, the physician can make an informed decision to decrease the dose of the therapeutic compound. Likewise, if the results of the in vitro assay with the patient's microbiome predict an increase in EHR, the physician can make an informed decision to further decrease the dose of the therapeutic compound.

### Treating

The method can further include treating the patient. In one or more embodiments, the use of the methods described herein can be used to adjust the dosage of an active compound so that the therapeutic window is achieved and/or maintained. For instance, the model may suggest that the amount of active compound circulated by EHR is high enough to result in toxic concentrations of the compound when it is administered systemically to the patient. In such a situation the dosage can be adjusted to administer less of the active compound. Alternatively, the model may suggest that the amount of active compound circulated by EHR is too low to be effective when it is administered systemically to the patient. In such a situation the dosage can be adjusted to administer more of the active compound.

Thus, in another aspect, this disclosure describes a method of identifying a subject as being a subject likely to respond weakly to a compound. Generally, the method includes administering the compound to the subject for a period of time sufficient to achieve steady state, detecting cells of the subject's microbiome that contain the active compound, and identifying the subject as a weak responder to the active compound. As used herein, the term “weak responder” encompasses a response from the subject that is less than the desired therapeutic response to any degree and, therefore, includes non-responders (those subjects who do not respond at all to the therapy). In this aspect, sequestering of the active compound-either in an active or inactive state-by cells in the microbiome reduces the amount of active compound circulated by EHR. Sequestering of an active compound that does not undergo EHR can also reduce the amount of the active compound in the blood stream. If there are reasons why an increased dose of the active compound may be undesirable (e.g., toxicity, patient compliance, etc.), the subject may be a candidate for an alternative therapy rather than continuing with therapy based on the active compound to which the subject responds more weakly than desired.

In another aspect, this disclosure describes a method of identifying a subject as being a subject likely to respond strongly to a compound. Generally, the method includes administering the compound to the subject for a period of time sufficient to achieve steady state, detecting cells of the subject's microbiome that contain lower than typical amount of the active compound, and identifying the subject as a strong responder to the active compound. As used herein, the term “strong responder” encompasses a response from the subject that is more than the desired therapeutic response to any degree and, therefore, includes those exhibiting toxicity of the compound (those subjects who have more side-effects of the therapy). In this aspect, reduced sequestering of the active compound-either in an active or inactive state-by cells in the microbiome increases the amount of active compound circulated by EHR. Reduced sequestering of an active compound that does not undergo EHR can also increase the amount of the active compound in the blood stream. Such a subject may be a candidate for a reduced dose of the active compound or an alternative therapy rather than continuing with therapy based on the active compound to which the subject responds more strongly than desired.

Releasing Sequestered Compound from Microbiome

In another aspect, this disclosure describes a method of releasing the sequestered drug from cells of the microbiome. Having detected that cells of the microbiome retain at least a portion of active compound, one may take additional steps to induce cells of the microbiome to release the sequestered therapeutic compound. Microbiome cells may be induced to release sequestered active compounds by disrupting the cells or otherwise inducing the cells to release the therapeutic compound. For example, the addition of MPAG to microbiome cells in the in vitro assay led to the release of MPA that had accumulated inside the cells. Cells of the microbiome may be disrupted by any method known to those of skill in the art to disrupt microbes. Exemplary methods for disrupting microbial cells include, but are not limited to, physical disruption (e.g., agitation), chemical or enzymatic disruption (e.g., lysozyme) or osmotic disruption (e.g., treatment with a hypotonic solution).

In one or more embodiments, the microbiome cells are microbiome cells of a subject that has received the therapeutic compound for a period of time sufficient to achieve steady state.

### Embodiments

The invention is defined in the claims. However, below there is provided a non-exhaustive listing of non-limiting exemplary aspects. Any one or more of the features of these aspects may be combined with any one or more features of another example, embodiment, or aspect described herein.

Embodiment 1. A method for evaluating metabolism of an active compound, comprising:
providing a composition comprising a microbiome, wherein the microbiome is obtained from a subject that has received an active compound for a period of time sufficient to achieve steady state;
combining a deactivated metabolite of the active compound and the composition to form a mixture;
incubating the mixture;
identifying a change in the mixture, wherein the change comprises a reduction in the amount of the deactivated metabolite, presence of a re-activated active compound, or both;
estimating, from the change in the mixture, the ability of the subject's microbiome to process the deactivated metabolite in the subject and affect the concentration of the active compound in the subject;
determining a dosage required to achieve a concentration of the active compound in the subject that is therapeutically effective considering effects of the subject's microbiome on the concentration of the active compound in the subject; and
administering to the subject the dosage required to achieve a concentration of the active compound in the subject that is therapeutically effective.
   Embodiment 2. The method of Embodiment 1, wherein the microbiome is a human microbiome.
   Embodiment 3. The method of Embodiment 1, wherein the active compound is a pro-drug.
   Embodiment 4. The method of Embodiment 1, wherein the metabolite is a metabolite of a drug or a metabolite of a pro-drug.
   Embodiment 5. The method of Embodiment 1, wherein incubating the mixture occurs for at least 1 hour, at least 2 hours, at least 3 hours, at least 4 hours, at least 5 hours, or at least 6 hours before identifying the change in the mixture.
   Embodiment 6. The method of Embodiment 1, further comprising obtaining an abbreviated AUC, a trough concentration, or a combination thereof, from the subject, wherein the estimating comprises using the change in the mixture and either the abbreviated AUC, the trough concentration, or the combination thereof.
   Embodiment 7. The method of Embodiment 1, wherein the active compound is a compound that is inactivated by the liver.
   Embodiment 8. A composition comprising:
a microbiome obtained from a subject that has received an active compound for a period of time sufficient to achieve steady state; and
a deactivated metabolite of the active compound.
   Embodiment 9. The method of Embodiment 1, further comprising:
screening a sample from the subject to detect the presence or absence of a polymorphic variant of a coding region encoding at least one of a hepatic enzyme, a transporter, or a combination thereof, and
determining variation in metabolism of the active compound by the at least one hepatic enzyme, transporter, or a combination thereof.
   Embodiment 10. The method of Embodiment 9, wherein the hepatic enzyme or transporter are one or more or a cytochrome P450 protein, UGT1A8/9, ABCB1, UGT2B7, SLCO1B1, or MRP-2.
   Embodiment 11. The method of Embodiment 9, wherein the estimating comprises using the change in the mixture and the variation in metabolism of the active compound.
   Embodiment 12. The method of Embodiment 9, wherein the active compound is a drug processed by a hepatic enzyme, a transporter, or a combination thereof, and subsequently undergoes metabolism by the microbiome.
   Embodiment 13. The method of Embodiment 9, wherein the polymorphic variant is associated with:
an increase or decrease in expression of a hepatic enzyme or a transporter;
an increase or decrease in activity of a hepatic enzyme or a transporter; or
an increase or decrease in hepatic metabolism of the active compound by a hepatic enzyme or a transporter and subsequently undergoes metabolism by the microbiome of the subject.
   Embodiment 14. A method for evaluating metabolism of a compound, the method comprising:
providing a composition comprising a microbiome, wherein the microbiome is obtained from a subject that has received an active compound for a period of time sufficient to achieve steady state;
combining the active compound and the composition to form a mixture;
incubating the mixture;
identifying a change in the mixture, wherein the change comprises a reduction in the amount of the active compound, presence of the active compound modified by the microbiome, or both;
estimating from the change in the mixture the ability of the subject's microbiome to process the active compound in the subject and affect the concentration of the active compound in the subject;
determining a dosage required to achieve a concentration of the active compound in the subject that is therapeutically effective considering effects of the subject's microbiome on the concentration of the active compound in the subject; and
administering to the subject the dosage required to achieve a concentration of the active compound in the subject that is therapeutically effective.
   Embodiment 15. The method of Embodiment 14, wherein the microbiome is a human microbiome.
   Embodiment 16. The method of Embodiment 14, wherein the active compound is a drug that is absorbed in the gastro-intestinal tract.
   Embodiment 17. The method of Embodiment 14, wherein the active compound is a drug administered orally and is modified by the microbiome prior to absorption by intestinal cells.
   Embodiment 18. The method of Embodiment 14, wherein incubating the mixture occurs for at least 1 hour, at least 2 hours, at least 3 hours, at least 4 hours, at least 5 hours, or at least 6 hours before identifying the change in the mixture.
   Embodiment 19. The method of Embodiment 14, further comprising obtaining an abbreviated AUC, a trough concentration, or a combination thereof, from the subject, wherein the estimating comprises using the change in the mixture and either the abbreviated AUC, the trough concentration, or the combination thereof.
   Embodiment 20. A composition comprising:
a microbiome obtained from a subject that has received an active compound for a period of time sufficient to achieve steady state; and
the active compound.
   Embodiment 21. A method for identifying a subject as a weak responder to an active compound, the method comprising:
administering the active compound to the subject for a period of time sufficient to achieve steady state;
detecting cells of the subject's microbiome that contain the active compound; and
identifying the subject as a weak responder to the active compound.
   Embodiment 22. A method comprising:
providing cells containing an active compound; and
lysing the cells, thereby releasing the active compound.
   Embodiment 23. The method of Embodiment 22, wherein the cells comprise microbiome cells from a subject that has received the active compound for a period of time sufficient to achieve steady state.
   Embodiment 24. A method comprising:
providing cells containing an active compound; and
contacting the cells with a compound that induces the cells to release the active compound.
   Embodiment 25. The method in Embodiment 24, wherein the cells comprise microbiome cells from a subject that has received the therapeutic compound for a period of time sufficient to achieve steady state.
   Embodiment 26. The method of Embodiment 24 or Embodiment 25, wherein the compound that induces the cells to release the active compound comprises a metabolic substrate of the cells.
   Embodiment 27. A method for identifying a subject as a strong responder to an active compound, the method comprising:
administering the active compound to the subject for a period of time sufficient to achieve steady state;
detecting cells of the subject's microbiome that contain lower than typical amount of the active compound; and
identifying the subject as a strong responder to the active compound.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one or more embodiments,” “an embodiment,” “certain embodiments,” “some embodiments,” a numbered Embodiment, etc., means that a particular feature, configuration, composition, or characteristic described in connection with the embodiment is included in at least one or more embodiments of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one or more embodiments may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

### Methods

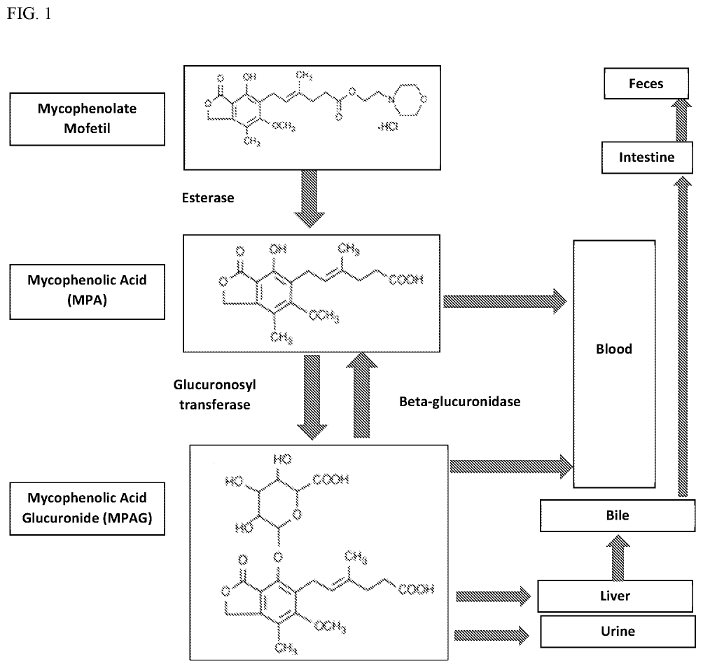

The following example used a stool sample of a kidney transplant patient who was on a steady state dose of 1 gram of mycophenolate mofetil (MMF) twice a day (FIG. 1). This patient underwent intensive pharmacokinetic sampling over 12 hours to assess enterohepatic recirculation (EHR) carefully and accurately. Once participants reach a steady state dose between days 15-45 post-transplant, fourteen blood samples were obtained at the time the morning MMF does was administered and at 30 minutes, one hour, two hours, three hours, four hours, five hours, six hours, seven hours, eight hours, nine hours, ten hours, 11 hours, and 12 hours following the morning MMF dose. The sampling strategy assured capture of the secondary peak and EHR characteristics.

A human microbiome sample from stool was collected from a patient between days 15-45 post-transplant, during their 12-hour pharmacokinetic visit. Prior to the initiation of the culture of microbiome stool sample, in the MPA arm of the experiment, a 1 mg/mL stock solution of MPA was diluted to 100 pg/mL in thirteen anaerobic culture tubes by adding 0.7 mL of the stock MPA solution to 6.3 mL of YCFA (Yeast extract-Casein hydrolysate-fatty acids) medium broth. Likewise, for the MPAG arm of the experiment, a 100 pg/mL stock solution of MPAG was diluted to 10 pg/mL in thirteen anaerobic culture tubes by adding 0.7 mL of the stock MPAG solution to 6.3 mL of YCFA (Yeast extract-Casein hydrolysate-fatty acids) medium broth.

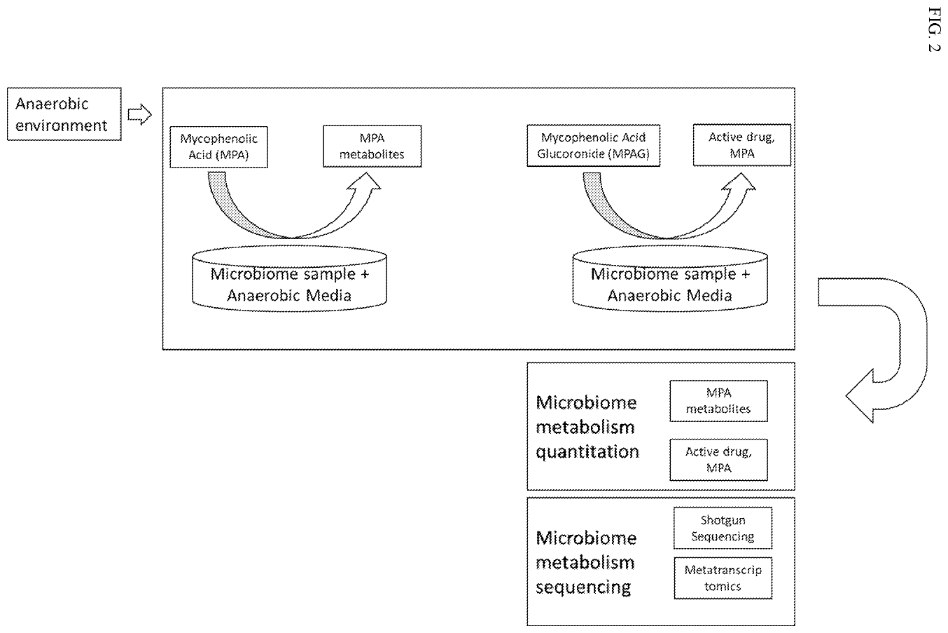

Stool aliquots of ˜0.5 g were incubated in separate YCFA tubes containing MPA or MPAG for six hours, 24 hours, or 48 hours in separate tubes, at 37° C. under anaerobic conditions (FIG. 2). All aliquots were collected at each timepoint in triplicate. Aliquots were also collected from MPA and MPAG tubes without any stool samples at zero, six hours, 24 hours, and 48 hours to serve as controls for this experiment.

After incubation, samples were vortexed at 16,000×g for 10 minutes, and the supernatant was collected and stored at −80° C. prior to high performance liquid chromatography-tandem mass spectrometry (HPLC-MS/MS) analysis. The supernatant was assessed for the presence and concentration of total MPA and MPAG using a validated mass spectrometry assay. Total MPA and MPAG concentration were determined based on certified analytical reference standards of 100 pg/mL of MPA and MPAG. These analytical reference standards were used as internal standards during the mass spectrometry assay.

### Results

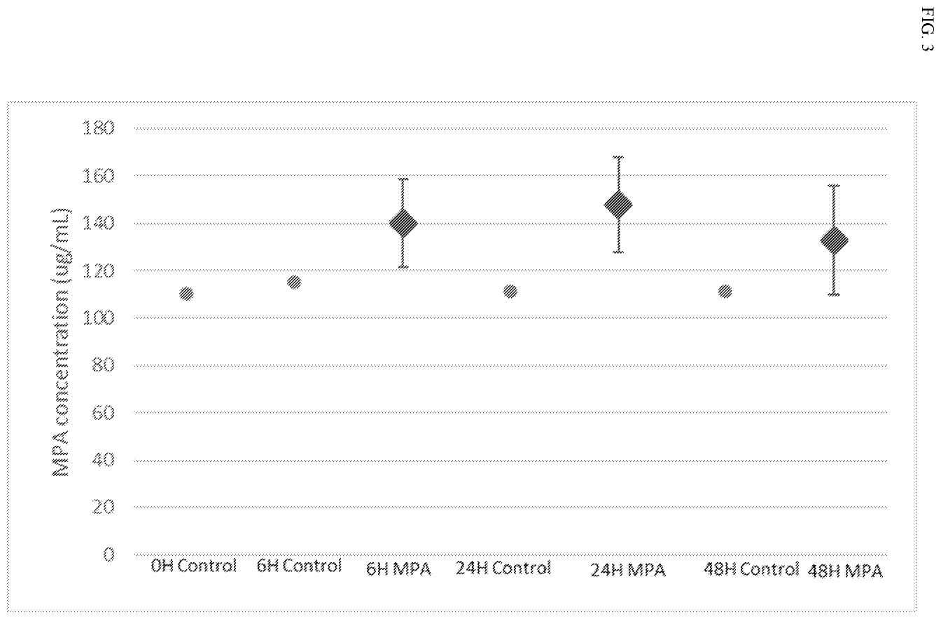

In the MPA arm of the experiment, no difference was detected in the concentration of MPA in control samples and in the MPA tubes incubated for six hours, 24 hours, or 48 hours with stool samples (FIG. 3). The control samples have no stool sample in them. This suggests that MPA was not being actively metabolized by the stool bacteria in our in vitro model. Further, there were no detectable concentrations of MPAG in the MPA tubes incubated with stool microbiome samples. This suggests that MPA was not converted to MPAG in the in vitro model.

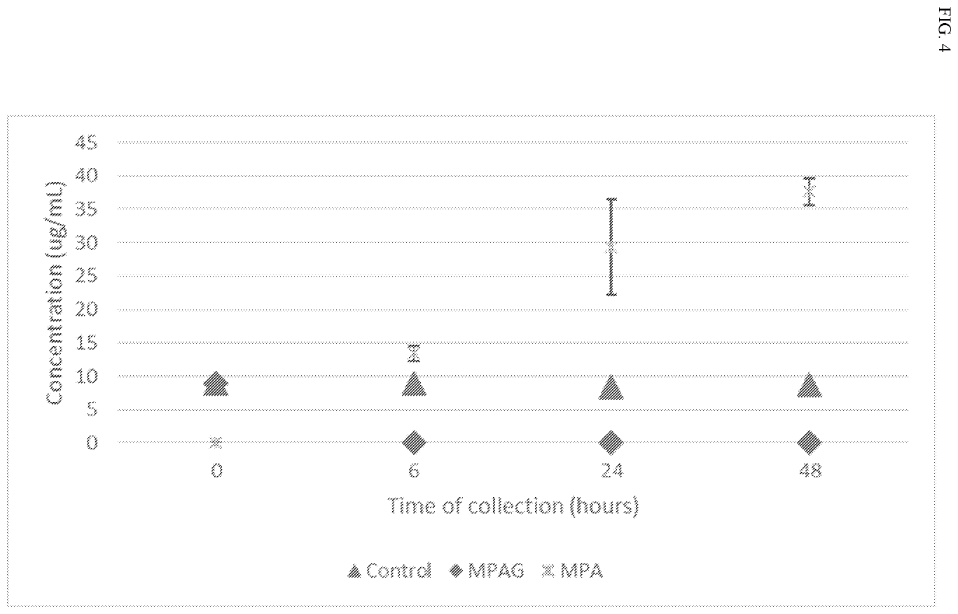

In the MPAG arm of the experiment, a rapid decrease in MPAG concentrations was observed in the MPAG tubes incubated with stool microbiome samples at six hours post incubation, while the MPAG concentration in the control aliquots remained stable over the duration of the experiment (FIG. 4). In addition, an increase was detected in the concentration of MPA starting at six hours after incubation, with a continuing upward trend in the 24-hour and 48-hour incubation tubes (FIG. 4). Taken together, the data from the MPAG arm of the experiment suggest that MPAG was metabolized by the stool microbiome into MPA in the in vitro model.

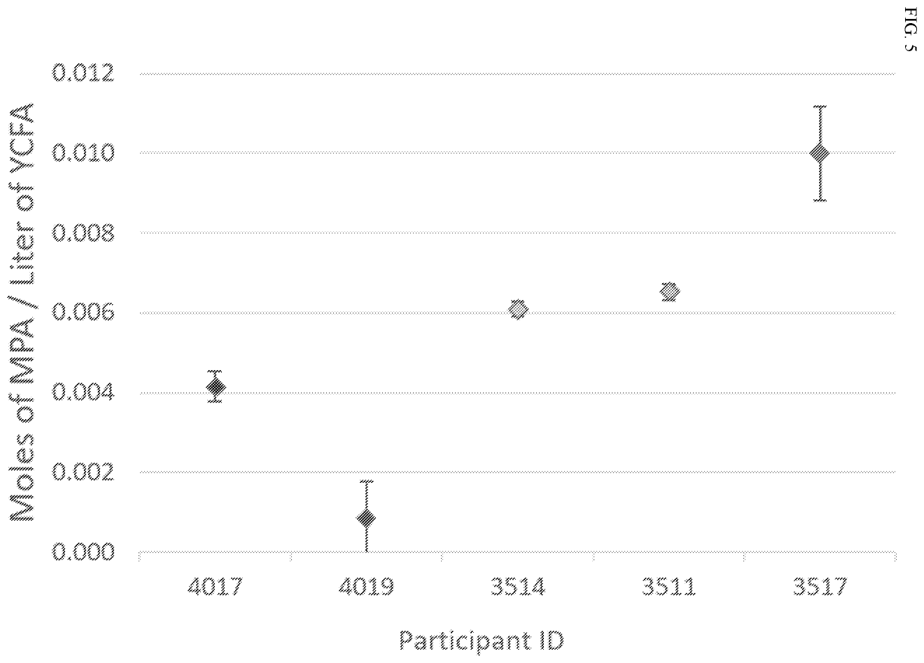

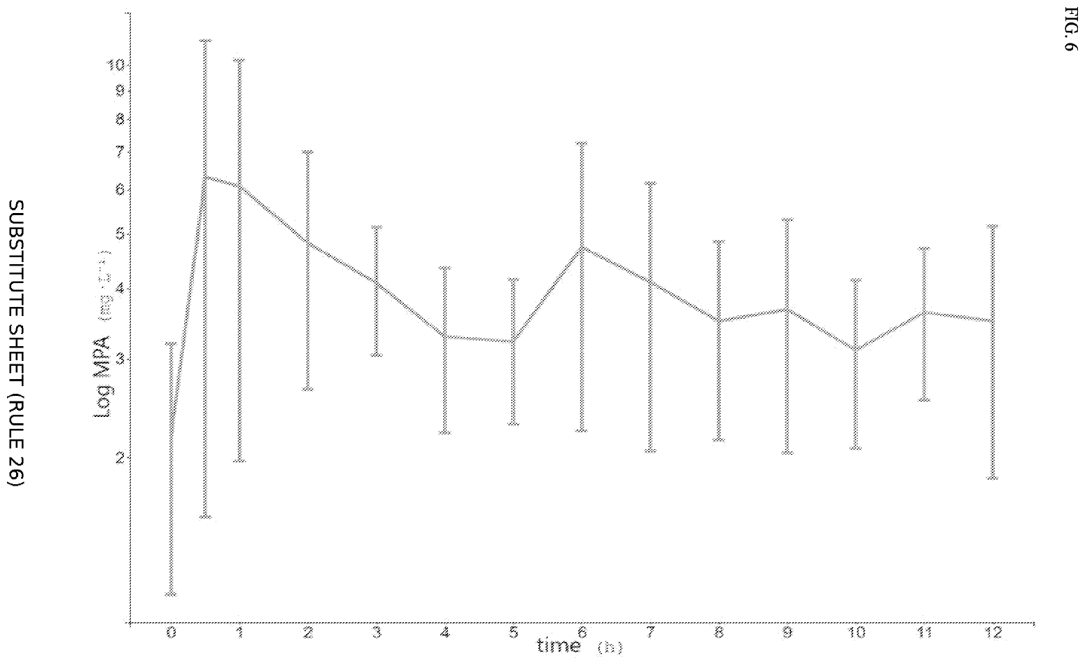

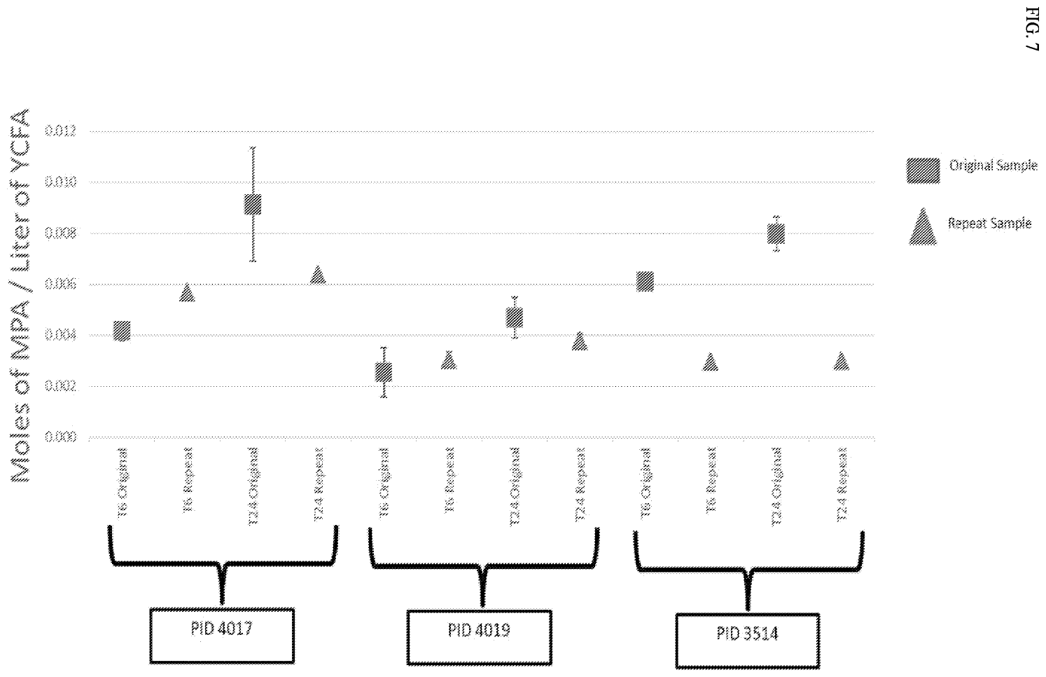

Five kidney transplant recipients taking MMF and tacrolimus as maintenance immunosuppression had their six-hour MPA levels assessed in the in vitro assay exposing microbiome to MPAG (FIG. 5). These patients also underwent an extensive 12-hour MPA pharmacokinetic (PK) profile (FIG. 6), and the stool samples were collected at the time of the PK profile. The percent EHR in these kidney transplant recipients was defined as [MPA AUC6-12 hour]/[AUC0-12 hour]×100 as a measure of their in vivo EHR. The correlation coefficient between the six-hour MPA level from the in vitro model with percentage EHR was 0.82 (95% CI—0.21 to 0.98, P=0.08). These five participants had high MPAG to MPA conversion in the in vitro assay compared to other participants, and the correlation may vary in other participants. Four of the five participants with high MPAG to MPA conversion had their stool sample collected at the time of pharmacokinetic study, frozen at −80° C. for a mean of 312 days (range 246-385 days), thawed at 4° C. overnight, then used for a repeat in vitro assay at 37° C. The repeat in vitro assay showed ability of the stool microbiome sample to metabolism MPAG to MPA as before (FIG. 7).

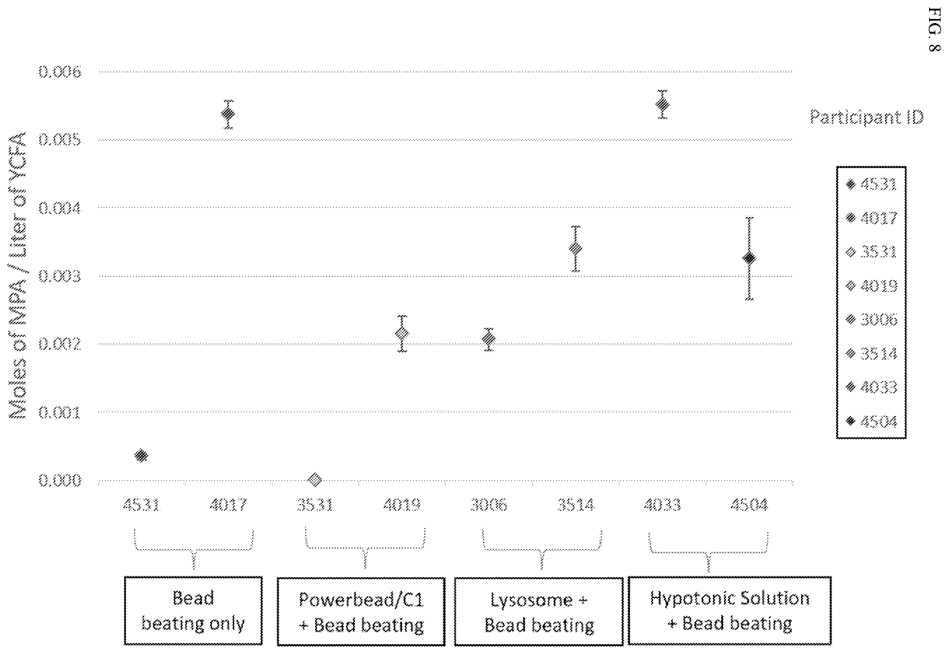

The in vitro assay can account for the MPA that has bioaccumulated in the stool microbes prior to the in vitro assay (FIG. 8) and at the end of the assay. This bioaccumulation can be determined by lysing bacteria from the stool microbiome sample aliquot obtained for the in vitro assay and determining if any MPA has been bioaccumulated. The stool samples collected from eight patients at the pharmacokinetic profile were treated using a combination of physical disruption (three cycles of 90 seconds of bead beating) with four different additives to facilitate cell lysis [750 μL of POWERBEAD solution (Qiagen, Hilden, Germany)+60 μL of C1 solution per the POWERSOIL (Qiagen, Hilden, Germany) microbiome protocol, 200 pg/ml of lysozyme+100 μL of proteinase inhibitor, or 10 mL of hypotonic solution+100 μL of proteinase inhibitor].

### Summary

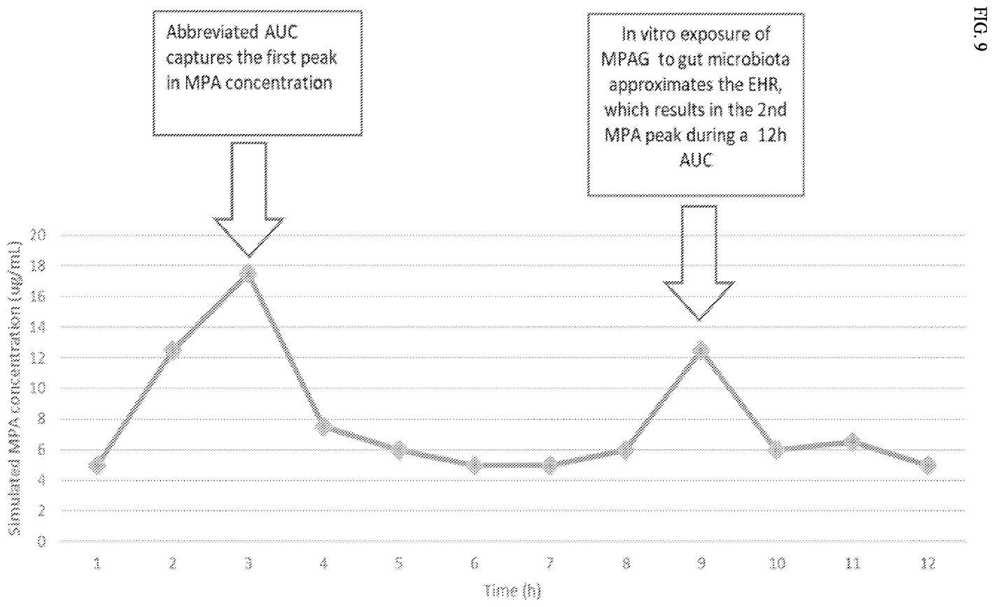

These preliminary data suggest that after exposing the stool microbiota to an inactive metabolite (MPAG) under anaerobic condition, an increase of the activated drug created by the microbes in the culture was detected. This in vitro assay approximates the EHR and the drug metabolism in vivo by the microbiome, whereas current methods of two-hour, three-hour, or four-hour abbreviated AUCs or trough concentration fail to account for EHR, despite its potential effect on the variability in MPA pharmacokinetics in the body (FIG. 9). Thus, the methods described herein allow one to determine the dose appropriate for individual patient's drug metabolism based on their microbiome anerobic culture rather than administering the dose based on other less individualized factors such as race or the general population. For example, African American patients are often prescribed higher doses of 1.5 grams twice per day.

The in vitro assay can account for the MPA that has bioaccumulated in the gut microbes prior to the in vitro assay. This bioaccumulation can be determined by lysing bacteria from the stool microbiome sample aliquot obtained for the in vitro assay and determining if any MPA has been bioaccumulated. The addition of MPAG to the microbiome also promotes the release of bioaccumulated MPA (FIG. 5 and FIG. 8).

### Example 2

### Methods

Genetic variants in the DNA of 480 participants, undergoing MPA trough level measurements for clinical reasons, were determined in a genome wide association study (GWAS) of MPA trough levels around 70 days post kidney transplant. The MPA trough levels across day 1 to day 180 post-transplant were analyzed and there was a spline time point around day 80 post-transplant, meaning the slope of the MPA trough trajectory changed. Several models with actual trough levels with different spline time points were used to determine which model fit the actual trough data best by comparing the maximized log-likelihood. The best model was with spline at day 70. Therefore, the analysis used the day 70 posttransplant predicted trough level as the outcome and all subjects were taking MMF and tacrolimus for immunosuppression at time of MPA level. The analysis was done using linear mixed models with random intercepts. A polygenic risk score was created based on association of genetic variants across the human genome (also known as GWAS), with the predicted MPA trough levels. The association was adjusted for covariates such as transplant recipient's age, gender and first five principal components. The adjustment for principal components was done to account for ethnicity of the transplant recipients in the study population. The SNPs were retained if p-value for the association if sample missingness was <0.1 (10%), minor allele frequency greater than 5%, and imputation information score was >80% and LD prune with r² less than 20%.

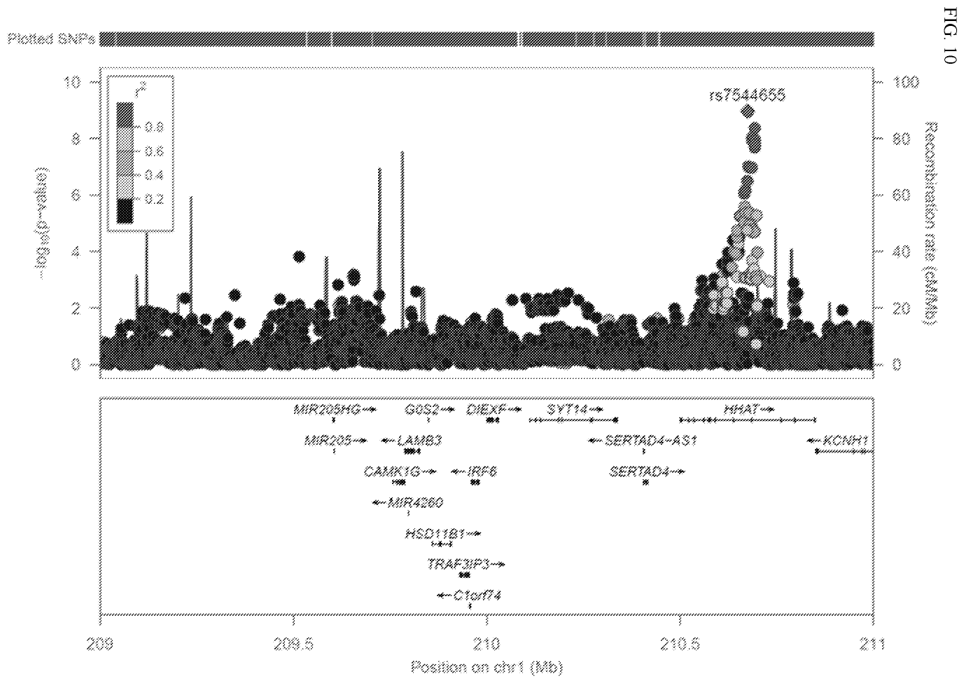

The most strongly associated SNPs with MPA trough levels are shown in Table 2. The SNP with the strongest association was rs7544655 in the HHAT gene. The regional plot showing SNP most strongly associated with predicted MPA trough level is in FIG. 10.

The SNPs with p-value<10⁻⁵ included in the polygenic risk score for association with MPA trough level are shown in Table 3. The weights in the polygenic risk score were determined by the effect size estimated from the genome wide association analysis.

TABLE 2
SNPs in the HHAT gene were most strongly associated with
MPA trough levels at 70 days post kidney transplant

| | | | A1 | | | | | | | |
|---|---|---|---|---|---|---|---|---|---|---|
| | | | frequency | | | | | Std. | | |
| SNP | Chr | position | (cohort) | info | A0 | A1 | Estimate | Error | t value | P-value |
| rs7544655 | 1 | 210675102 | 0.728 | 1 | C | T | 0.11512 | 0.018498 | 6.223429 | 1.08 × 10⁻⁹ |
| rs4545340 | 1 | 210686570 | 0.735 | 0.987 | A | G | 0.11032 | 0.018837 | 5.856505 | 9.09 × 10⁻⁹ |
| rs12049235 | 1 | 210687044 | 0.733 | 0.989 | G | C | 0.110299 | 0.019 | 5.805293 | 1.21 × 10⁻⁸ |
| rs10863839 | 1 | 210689796 | 0.741 | 0.984 | T | C | 0.11355 | 0.019374 | 5.860876 | 8.92 × 10⁻⁹ |
| rs71571953 | 1 | 210693036 | 0.695 | 0.983 | AT | A | 0.110289 | 0.018947 | 5.820907 | 1.11 × 10⁻⁸ |
| rs11119519 | 1 | 210693399 | 0.694 | 0.981 | A | G | 0.109664 | 0.019003 | 5.770947 | 1.47 × 10⁻⁸ |
| rs10863840 | 1 | 210693445 | 0.694 | 0.981 | T | C | 0.108294 | 0.018947 | 5.715708 | 2.00 × 10⁻⁸ |
| rs10746426 | 1 | 210693516 | 0.735 | 0.981 | T | C | 0.114914 | 0.019168 | 5.995075 | 4.18 × 10⁻⁹ |
| rs11119521 | 1 | 210693847 | 0.695 | 0.982 | T | G | 0.110254 | 0.01895 | 5.818261 | 1.13 × 10⁻⁸ |
| rs11119522 | 1 | 210694001 | 0.695 | 0.982 | T | G | 0.110245 | 0.018951 | 5.817406 | 1.14 × 10⁻⁸ |
| rs11119523 | 1 | 210694614 | 0.695 | 0.98 | C | T | 0.108867 | 0.018907 | 5.758051 | 1.58 × 10⁻⁸ |

Chr = Chromosome; A1 frequency = minor allele frequency; SNPs with info score >80% indicating level of imputation certainty; A0 = allele genotype; A1 = corresponding allele to A0; Estimate = estimated effect size; Std = Standard error; t-value = level of statistical significance.

TABLE 3
SNPs in the polygenic risk score associated with MPA trough levels at 70 days post kidney transplant.

| | | | A1 | | | | | Std. | | |
|---|---|---|---|---|---|---|---|---|---|---|
| SNP | Chr | position | frequency | info | A0 | A1 | Estimate | Error | t value | P value |
| rs12097411 | 1 | 151000257 | 0.153 | 0.911 | T | C | −0.12178 | 0.026815 | −4.54144 | 7.37 × 10⁻⁶ |
| rs2095135 | 1 | 170308646 | 0.357 | 0.992 | C | T | 0.079069 | 0.017503 | 4.517519 | 7.97 × 10⁻⁶ |
| rs7546029 | 1 | 210665251 | 0.637 | 0.995 | T | C | 0.075234 | 0.016662 | 4.515435 | 8.01 × 10⁻⁶ |
| rs12121566 | 1 | 210679381 | 0.59 | 0.993 | C | T | 0.081498 | 0.017491 | 4.659306 | 4.16 × 10⁻⁶ |
| rs10863837 | 1 | 210683732 | 0.538 | 0.996 | A | G | 0.076173 | 0.017006 | 4.479117 | 9.44 × 10⁻⁶ |
| rs7536785 | 1 | 233648237 | 0.514 | 0.936 | T | C | −0.08551 | 0.018511 | −4.61955 | 5.27 × 10⁻⁶ |
| rs73101509 | 1 | 233655461 | 0.267 | 0.998 | A | G | −0.089 | 0.019213 | −4.63221 | 4.70 × 10⁻⁶ |
| rs143287113 | 2 | 138410676 | 0.109 | 0.955 | T | TAAGAG | −0.1335 | 0.028403 | −4.70016 | 3.48 × 10⁻⁶ |
| rs305211 | 2 | 36272521 | 0.791 | 0.99 | C | T | 0.097003 | 0.02148 | 4.516057 | 8.00 × 10⁻⁶ |
| rs7768704 | 6 | 14118998 | 0.383 | 0.973 | G | A | 0.085585 | 0.017439 | 4.907738 | 1.31 × 10⁻⁶ |
| rs2147819 | 6 | 164694679 | 0.086 | 1 | T | C | −0.1412 | 0.031405 | −4.49608 | 8.73 × 10⁻⁶ |
| rs9259439 | 6 | 29864240 | 0.638 | 0.928 | T | C | 0.101388 | 0.02036 | 4.979711 | 9.53 × 10⁻⁷ |
| rs4745805 | 10 | 77767494 | 0.373 | 0.973 | T | C | 0.087806 | 0.01895 | 4.63364 | 4.73 × 10⁻⁶ |
| rs761988 | 14 | 24980685 | 0.28 | 0.999 | T | C | 0.091272 | 0.019334 | 4.720822 | 3.11 × 10⁻⁶ |
| rs71350694 | 20 | 49256566 | 0.126 | 0.95 | G | A | −0.12719 | 0.027317 | −4.65615 | 4.27 × 10⁻⁶ |

Chr = Chromosome;
A1 frequency = minor allele frequency;
SNPs with info score >80% indicating level of imputation certainty;
A0 = allele genotype;
A1 = corresponding allele to A0;
Estimate = estimated effect size;
Std = Standard error;
t-value = level of statistical significance

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for evaluating metabolism of an active compound, comprising:
providing a composition comprising a microbiome, wherein the microbiome is obtained from a subject that has received an active compound for a period of time sufficient to achieve steady state;
combining a deactivated metabolite of the active compound and the composition to form a mixture;
incubating the mixture;
identifying a change in the mixture, wherein the change comprises a reduction in the amount of the deactivated metabolite, presence of a re-activated active compound, or both;
estimating, from the change in the mixture, the ability of the subject's microbiome to process the deactivated metabolite in the subject and affect the concentration of the active compound in the subject;
determining a dosage required to achieve a concentration of the active compound in the subject that is therapeutically effective considering effects of the subject's microbiome on the concentration of the active compound in the subject; and
administering to the subject the dosage required to achieve a concentration of the active compound in the subject that is therapeutically effective.

2. The method of claim 1, wherein the microbiome is a human microbiome.

3. The method of claim 1, wherein the active compound is a pro-drug.

4. The method of claim 1, wherein the metabolite is a metabolite of a drug or a metabolite of a pro-drug.

5. The method of claim 1, wherein incubating the mixture occurs for at least 1 hour, at least 2 hours, at least 3 hours, at least 4 hours, at least 5 hours, or at least 6 hours before identifying the change in the mixture.

6. The method of claim 1, further comprising obtaining an abbreviated AUC, a trough concentration, or a combination thereof, from the subject, wherein the estimating comprises using the change in the mixture and either the abbreviated AUC, the trough concentration, or the combination thereof.

7. The method of claim 1, wherein the active compound is a compound that is inactivated by the liver.

8. A composition comprising:
a microbiome obtained from a subject that has received an active compound for a period of time sufficient to achieve steady state; and
a deactivated metabolite of the active compound.

9. The method of claim 1, further comprising:
screening a sample from the subject to detect the presence or absence of a polymorphic variant of a coding region encoding at least one of a hepatic enzyme, a transporter, or a combination thereof; and
determining variation in metabolism of the active compound by the at least one hepatic enzyme, transporter, or a combination thereof.

10. The method of claim 9, wherein the hepatic enzyme or transporter are one or more or a cytochrome P450 protein, UGT1A8/9, ABCB1, UGT2B7, SLCO1B1, or MRP-2.

11. The method of claim 9, wherein the estimating comprises using the change in the mixture and the variation in metabolism of the active compound.

12. The method of claim 9, wherein the active compound is a drug processed by a hepatic enzyme, a transporter, or a combination thereof, and subsequently undergoes metabolism by the microbiome.

13. The method of claim 9, wherein the polymorphic variant is associated with:
an increase or decrease in expression of a hepatic enzyme or a transporter;
an increase or decrease in activity of a hepatic enzyme or a transporter; or
an increase or decrease in hepatic metabolism of the active compound by a hepatic enzyme or a transporter and subsequently undergoes metabolism by the microbiome of the subject.

14. A method for evaluating metabolism of a compound, the method comprising:
providing a composition comprising a microbiome, wherein the microbiome is obtained from a subject that has received an active compound for a period of time sufficient to achieve steady state;
combining the active compound and the composition to form a mixture;
incubating the mixture;
identifying a change in the mixture, wherein the change comprises a reduction in the amount of the active compound, presence of the active compound modified by the microbiome, or both;
estimating from the change in the mixture the ability of the subject's microbiome to process the active compound in the subject and affect the concentration of the active compound in the subject;
determining a dosage required to achieve a concentration of the active compound in the subject that is therapeutically effective considering effects of the subject's microbiome on the concentration of the active compound in the subject; and
administering to the subject the dosage required to achieve a concentration of the active compound in the subject that is therapeutically effective.

15. The method of claim 14, wherein the microbiome is a human microbiome.

16. The method of claim 14, wherein the active compound is a drug that is absorbed in the gastro-intestinal tract.

17. The method of claim 14, wherein the active compound is a drug administered orally and is modified by the microbiome prior to absorption by intestinal cells.

18. The method of claim 14, wherein incubating the mixture occurs for at least 1 hour, at least 2 hours, at least 3 hours, at least 4 hours, at least 5 hours, or at least 6 hours before identifying the change in the mixture.

19. The method of claim 14, further comprising obtaining an abbreviated AUC, a trough concentration, or a combination thereof, from the subject, wherein the estimating comprises using the change in the mixture and either the abbreviated AUC, the trough concentration, or the combination thereof.

20. A composition comprising:
a microbiome obtained from a subject that has received an active compound for a period of time sufficient to achieve steady state; and
the active compound.

21. A method for identifying a subject as a weak responder to an active compound, the method comprising:
administering the active compound to the subject for a period of time sufficient to achieve steady state;
detecting cells of the subject's microbiome that contain the active compound; and
identifying the subject as a weak responder to the active compound.

22. A method comprising:
providing cells containing an active compound; and
lysing the cells, thereby releasing the active compound.

23. The method of claim 22, wherein the cells comprise microbiome cells from a subject that has received the active compound for a period of time sufficient to achieve steady state.

24. A method comprising:
providing cells containing an active compound; and
contacting the cells with a compound that induces the cells to release the active compound.

25. The method in claim 24, wherein the cells comprise microbiome cells from a subject that has received the therapeutic compound for a period of time sufficient to achieve steady state.

26. The method of claim 24, wherein the compound that induces the cells to release the active compound comprises a metabolic substrate of the cells.

27. A method for identifying a subject as a strong responder to an active compound, the method comprising:
administering the active compound to the subject for a period of time sufficient to achieve steady state;
detecting cells of the subject's microbiome that contain lower than typical amount of the active compound; and
identifying the subject as a strong responder to the active compound.
